(19) United States
(12) Patent Application Publication
(10) Pub. No.: US 2010/0292623 A1
(43) Pub. Date: Nov. 18, 2010

(54) POLYETHYLENIMINE NANOPARTICLE-CONTAINING MICROBICIDAL ELECTROSPUN POLYMER FIBERS FOR TEXTILE APPLICATIONS

(75) Inventors: Andreas Greiner, Amoneburg (DE); Thorsten Röcker, Gemunden/Felda (DE)

Correspondence Address: SPECKMAN LAW GROUP PLLC, 1201 THIRD AVENUE, SUITE 330, SEATTLE, WA, 98101, US

(73) Assignee: SCHOELLER TEXTIL AG, Sevelen (CH)

(21) Appl. No.: 12/446,749

(22) PCT Filed: Oct. 17, 2007
(86) PCT No.: PCT/CH2007/000509

§ 371 Date: Feb. 10, 2010

(30) Foreign Application Priority Data
Oct. 23, 2006 (CH) 1688/06
Oct. 17, 2007 (CH) PCT/CH2007/000509

(51) Int. Cl. A41D 31/00; D01D 1/02; D01D 5/06; D01D 5/10; B05D 1/00; D04H 1/00; C08L 1/00; A61B 19/08; A61F 13/00; A41D 13/12

(52) U.S. Cl. 602/42; 264/465; 427/180; 428/221; 442/327; 525/186; 128/849; 2/243.1; 2/456

(57) ABSTRACT

The present invention relates to a process for producing electrospun fibers comprising polyethyleneimine nanoparticles (PEIN). The use of PEIN permits the antibacterial finishing of electrospinnable polymers, provided that these polyethyleneimine nanoparticles are particles of derivatized polyethyleneimine (PEI), since pure underivatized PEI has no antibacterial action. Preference is given to using quaternized polyethyleneimine. The electrospinnable polymers can be coated with PEIN during and/or after the electrospinning. The polymeric fibers obtainable by the process according to the invention can be used for textile fibers, for example for the production of fibers for functional apparel or for fibrous nonwoven webs or fibrous mats for cell culture substrates.

### DESCRIPTION AND INTRODUCTION TO THE GENERAL FIELD OF THE INVENTION

The present invention relates to the fields of macromolecular chemistry, process technology, and textile and material sciences.

### STATE OF THE ART

For the production of nano- and mesofibers, the person skilled in the art is aware of a multitude of processes, among which electrospinning is currently of the greatest significance. In this process, which is described, for example, by D. H. Reneker, H. D. Chun in Nanotechn. 7 (1996), page 216 ff., a polymer melt or a polymer solution is typically exposed to a high electrical field at an edge which serves as an electrode. This can be achieved, for example, by extrusion of the polymer melt or polymer solution in an electrical field under low pressure through a cannula connected to one pole of a voltage source. Owing to the resulting electrostatic charging of the polymer melt or polymer solution, there is a material flow directed toward the counterelectrode, which solidifies on the way to the counterelectrode. Depending on the electrode geometries, nonwovens or ensembles of ordered fibers are obtained by this process. Whereas only fibers with diameters greater than 1000 nm have been obtained to date with polymer melts, it is possible to produce fibers with diameters greater than or equal to 5 nm from polymer solutions.

The prior art includes some processes for producing polymer fibers by means of electrospinning:

DE 10 2004 009 887 A1 relates to a process for producing fibers with a diameter of <50 μm by electrostatic spinning or spraying of a melt of at least one thermoplastic polymer.

DE 101 33 393 A1 discloses a process for producing hollow fibers with an internal diameter of 1 to 100 nm, in which a solution of a water-insoluble polymer—for example poly-L-lactide solution in dichloromethane or a nylon 46 solution in pyridine—is electrospun. A similar process is also known from WO 01/09414 A1 and DE 103 55 665 A1.

DE 196 00 162 A1 discloses a process for producing lawnmower wire or textile fabrics, in which polyamide, polyester or polypropylene as a thread-forming polymer, a maleic anhydride-modified polyethylene/polypropylene rubber and one or more ageing stabilizers are combined, melted and mixed with one another, before this melt is melt-spun.

For some fields of application of fibers, it is desirable to be able to inhibit the growth and/or the proliferation of microorganisms. Microorganisms are understood to mean bacteria, fungi, algae, protozoa and viruses. Fibers with microbicidal properties are intended for use particularly in the medical sector, for example for wound dressings or textiles for patients and medical personnel. Hereinafter, unless explicitly stated otherwise, the terms “microbicide” and “microbicidal” are used as collective terms, respectively, for means of controlling microorganisms and for an antimicrobial action. The action against the microorganisms may be reversibly or irreversibly growth-inhibiting (for example bacteriostats or fungistats) or lethal (for example bactericides or fungicides).

The person skilled in the art will be aware that some organic nitrogen compounds have microbicidal properties.

For instance, DE 32 37 074 A1 describes polymer biguanides which can be used as microbicides in disinfectants. The polymer biguanides inhibit, for example, the growth of Aspergillus niger, Escherichia coli, Staphylococcus aureus, Pseudomonas aeruginosa and Chaetonium globosum.

DE 33 14 294 A1 describes condensed polyalkyleneimine polymers, with the aid of which biological materials such as whole cells and enzymes can be immobilized. To this end, the polyalkyleneimines are condensed together with a dicarboxylic acid to give a copolymer. Optionally, the copolymer is subsequently aftertreated with an amine crosslinking component. However, no derivatized polyalkyleneimines are used.

DE 34 23 703 A1 describes polymeric quaternary ammonium compounds which are obtained by reaction of polymers of the ionene type with tertiary amines. These inventive polymeric quarternary ammonium compounds have microbicidal properties. Additionally described are processes for inhibiting the growth and the proliferation of microorganisms, wherein the microorganisms are contacted with the inventive polymeric quarternary ammonium compounds. However, these polymeric quaternary ammonium compounds have no crosslinking of the polymer chains, and it is pointed out explicitly that polyethyleneimines are not good microbicides.

N. Beyth et al., Biomaterials 27, 2006, 3995-4002 describes production and use of ammonium-polyethylene nanoparticles in composites for dentistry. For this purpose, polyethyleneimine (PEI) is crosslinked with dibromopentane in the first step, the crosslinked PEI is alkylated with bromooctane in the second step, and the secondary or tertiary amino groups of the alkylated and crosslinked PEI are quaternized with methyl iodide in the third step. The PEI particles obtained in this way were added to composite resins for dental fillings and incubated with the oral bacterium Streptococcus mutans. The PEI particles inhibited bacterial growth over a period of one month. However, it was not possible to incorporate the PEI particles permanently into the composite material.

To date, the prior art does not include a process for finishing textile fibers permanently or temporarily with polyethyleneimine particles, thus imparting microbicidal action to the fibers.

### Object

It is an object of the present invention to provide polymeric fibers having microbicidal properties and processes for production thereof.

### Achievement of the Object

The object of providing polymeric fibers having microbicidal properties is achieved in accordance with the invention by polymeric fibers comprising at least one electrospinnable polymer and nanoparticles comprising quaternized polyethyleneimine.

According to the invention, the at least one electrospinnable polymer is selected from the group consisting of poly-(p-xylylene); polyvinylidene halides, polyesters such as polyethylene terephthalate, polybutylene terephthalate; polyethers; polyolefins such as polyethylene, polypropylene, poly(ethylene/propylene) (EPDM); polycarbonates; polyurethanes; natural polymers, e.g. rubber; polycarboxylic acids; polysulfonic acids; sulfated polysaccharides; polylactides; polyglycosides; polyamides; homo- and copolymers of aromatic vinyl compounds such as poly(alkyl)styrenes, e.g. polystyrenes, poly-alpha-methyl styrenes; polyacrylonitriles, polymethacrylonitriles; polyacrylamides; polyimides; polyphenylenes; polysilanes; polysiloxanes; polybenzimidazoles; polybenzothiazoles; polyoxazoles; polysulfides; polyester amides; polyarylene vinylenes; polyether ketones; polyurethanes, polysulfones, inorganic-organic hybrid polymers such as ORMOCER® from the Fraunhofer Gesellschaft zur Förderung der angewandten Forschung e.V. Munich; silicones; wholly aromatic copolyesters; poly(alkyl)acrylates; poly(alkyl)methacrylates; polyhydroxyethyl methacrylates; polyvinyl acetates, polyvinyl butyrates; polyisoprene; synthetic rubbers such as chlorobutadiene rubbers, e.g. Neopren® from DuPont; nitrilebutadiene rubbers, e.g. Buna N®; polybutadiene; polytetrafluoroethylene; modified and unmodified celluloses, homo- and copolymers of alpha-olefins and copolymers constructed of two or more monomer units forming the aforementioned polymers; polyvinyl alcohols, polyalkylene oxides, for example polyethylene oxides; poly-N-vinylpyrrolidone; hydroxymethylcelluloses; maleic acids, alginates; collagens.

All aforementioned polymers can be used in the inventive polymeric fibers having microbicidal properties in each case individually or in any desired combinations with one another, and in any desired mixing ratio.

According to the invention, the nanoparticles comprise derivatized, preferably quaternized, polyethyleneimine of the general formula

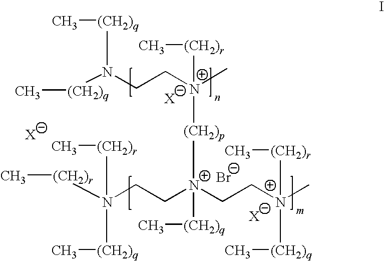

where
m and n are each independently a natural number from 5 to 200,
p is a natural number from 4 to 6,
q is a whole number from 0 to 11, and
r is a whole number from 0 to 4 and where
X is Br or I.

The object of providing a process for producing polymeric fibers having microbicidal properties, comprising at least one electrospinnable polymer and nanoparticles comprising quaternized polyethyleneimine, is achieved in accordance with the invention by a process comprising the steps of
a) crosslinking polyethyleneimine,
b) alkylating crosslinked polyethyleneimine,
c) quaternizing secondary and tertiary amino groups of the polyethyleneimine,
d) removing the quaternized polyethyleneimine nanoparticles,
e) adding the polyethyleneimine nanoparticles to a solution of one or more electrospinnable polymers,
f) electrospinning the solution of one or more electrospinnable polymers which contains polyethyleneimine nanoparticles to form fibers.

The process steps are illustrated in detail hereinafter:

### a) Crosslinking of Polyethyleneimine (PEI)

The crosslinking is performed according to the following scheme:

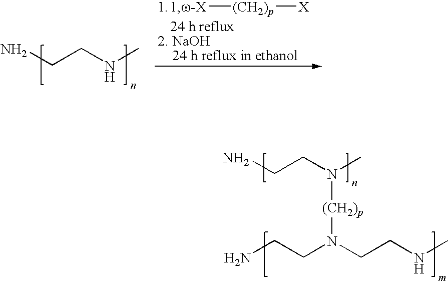

where n, m, p and X are each as defined above.

A commercial aqueous polyethyleneimine solution is completely dewatered by refluxing in toluene using a water separator. The anhydrous PEI is subsequently reacted with a linear unbranched 1,ω-dihaloalkane having 4 to 6 carbon atoms as a crosslinker, the halogen being bromine or iodine. The dihaloalkanes for use as crosslinkers are accordingly selected from 1,4-dibromobutane, 1,4-diiodobutane, 1-5-dibromopentane, 1,5-diiodopentane, 1-6-dibromohexane and 1,6-diiodohexane.

### b) Alkylation of Crosslinked Polyethyleneimine

The crosslinked polyethyleneimine is alkylated according to the scheme

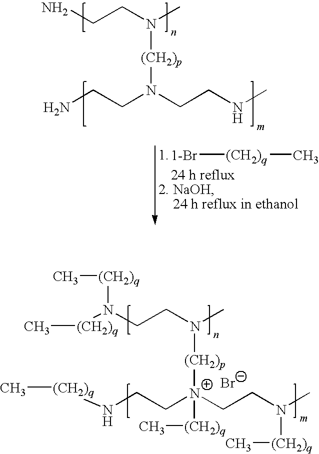

where n, m, p and q are each as defined above.

The crosslinked PEI is alkylated with a linear unbranched 1-bromoalkane having 1 to 12 carbon atoms. The alkylation is preferably effected with 1-bromoalkanes having 7 to 9 carbon atoms, i.e. 1-bromoheptane, 1-bromooctane or 1-bromononane.

### c) Quaternization of Secondary and Tertiary Amino Groups of the Polyethyleneimine

The secondary and tertiary amino groups of the polyethyleneimine are quaternized according to the scheme

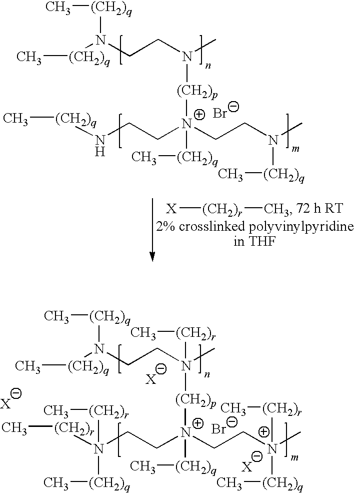

where m, n, q, r and X are each as defined above.

For the quaternization, the alkylated crosslinked PEI is reacted with a linear unbranched 1-haloalkane having 1 to 5 carbon atoms, the halogen being bromine or iodine. For the quaternization, preference is given to using an iodoalkane, more preferably methyl iodide. Polyvinylpyridine is used in this reaction as a proton sponge.

### d) Removal of the Quaternized Polyethyleneimine Nanoparticles

The polyethyleneimine nanoparticles obtained after performance of steps a) to c) are obtained in the form of a powder and can be removed from the reaction mixture, for example, by filtration. The resulting PEI nanoparticles are readily dispersible in tetrahydrofuran (THF), ethanol and formic acid.

### e) Addition of the Polyethyleneamine Nanoparticles to a Solution of One or More Electrospinnable Polymers

One or more electrospinnable polymers are dissolved, preferably in THF, ethanol or formic acid, and then polyethyleneimine nanoparticles are added. Preference is given to preparing those solutions which contain 5% by weight to 25% by weight of the electrospinnable polymer and 0.01% by weight to 5% by weight of polyethyleneimine nanoparticles.

f) Electrospinning of the Solution of One or More Electrospinnable Polymers which Contains Polyethyleneimine Nanoparticles to Form Fibers

This solution is exposed to a high electrical field at an edge serving as an electrode. For example, this can be done by extruding the solution of the electrospinnable polymer containing polyethyleneimine nanoparticles in an electrical field under low pressure through a cannula connected to one pole of a voltage source. There is a material flow directed toward the counterelectrode, which solidifies on the way to the counterelectrode.

Alternatively, the solution of the one or more electrospinnable polymers can also first be spun to fibers without adding polyethyleneimine nanoparticles to the spinning solution. In this case, the electrospun polymer fibers are subsequently coated with PEI nanoparticles, according to the following process steps:
a) crosslinking polyethyleneimine,
b) alkylating crosslinked polyethyleneimine,
c) quaternizing secondary and tertiary amino groups of the polyethyleneimine,
d) removing the quaternized polyethyleneimine nanoparticles,
e) adding the polyethyleneimine nanoparticles to a solution of at least one electrospinnable polymer,
f) coating the electrospun fibers with polyethyleneimine nanoparticles.

The subsequent coating of the electrospun fibers with polyethyleneimine nanoparticles can be effected, for example, but not exclusively, by gas phase deposition, knife-coating, spin-coating, dip-coating, spraying or plasma deposition. These methods are known to those skilled in the art and can be used without leaving the scope of protection of the claims.

Optionally, the polyethyleneimine nanoparticles can either be spun to fibers together with the one or more electrospinnable polymers or be used for subsequent coating of the fibers.

In the inventive polymeric fibers having microbicidal properties, the proportion of the polyethyleneimine nanoparticles is 0.1% by weight to 25% by weight.

The inventive polymeric fibers with microbicidal properties inhibit the growth and/or the proliferation of microorganisms. Microorganisms are understood to mean bacteria, fungi, algae, protozoa and viruses.

The microbicidal polymeric fibers obtainable by the process according to the invention can be used to produce textile fibers and textile fabrics, for example for the production of fibers for textile fabrics for the production of functional apparel, protective apparel for medical personnel and protective apparel for patients, and also for surgical drapes and wound dressings or for fibrous nonwoven webs or fibrous mats for cell culture substrates.

### WORKING EXAMPLES

### 1. Production of Polyethyleneimine Nanoparticles

Polyethyleneimine nanoparticles were produced as described under “Achievement of the object”. In this case, 1,5-dibromopentane was used as the crosslinking agent in the first reaction step. In the second reaction step—the alkylation of the crosslinked PEI—2-bromooctane was used. In the third reaction step—the quaternization of the secondary and tertiary amino groups of the PEI—methyl iodide in THF was used.

### 2. Production of Antibacterial Nanofibers Based on PVB

The ethanol-soluble polymer polyvinyl butyrate (PVB, trade name Mowital) was used. Repeat unit of polyvinyl butyrate:

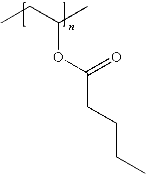

Polyvinyl butyrate (M_{w}=19 640, Mₙ=159 000, M_{w}/Mₙ=1.23) was dissolved in ethanol with stirring at room temperature. The concentrations of the solutions produced were 10 wt % and 15 wt %. In order to give the fibers an antibacterial finish, in each case 2 wt % of quaternized PEI particles were added to the polymer solutions and dispersed in the polymer solutions at room temperature with stirring.

The PVB dispersions were subsequently electrospun. The following parameters were set on the electrospinning system:

voltages: 15 kV, 20 kV, 25 kV, 30 kV
distance between cannula and electrode: 20 cm
cannula diameter: 0.3 mm
flow rates: 0.86 ml/h, 1.21 ml/h, 1.56 ml/h

The substrates used were aluminum foil and frames of aluminum sheet.

The resulting fibers had an average diameter of 1.3 to 1.5 μm.

This diameter is quite high for fibers produced by electrospinning, but can be explained by the high viscosity and the low electrical conductivity of the solution.

Both parameters are shown in table 1. The finished fibers exhibited a pale yellow color. This already indicates, without further study, that the yellow nanoparticles have been incorporated into the fibers or else adsorbed on the fibers. Under the SEM (FIG. 3), the fibers appear smooth and without any extraneous bodies adsorbed on the surface.

TABLE 1
characterization of the PVB solution used in EtOH

| PVB | | | Electrical | Surface |
|---|---|---|---|---|
| concentration/ | Viscosity/ | Content of PEI | conductivity/ | tension/ |
| wt % | Pa · s | particles/wt % | μS/cm | mN/m |
| 15 | 1.399 | 2 | 4.98 | 22.98 |

Since visual confirmation of the presence of the particles was impossible, an EDX study of the fibers produced was done. The spectrum in FIG. 4 shows, as well as the signal for carbon and oxygen, only a clear signal for iodine. Iodine in these amounts can, however, only have got into the fibers as a counter ion to the quaternary ammonium ions in the PEI particles.

Since the iodine is present as a counter ion to the quaternary ammonium ions in the PEI particles and cannot be separate therefrom, these particle must be present either in the fibers or on the fibers. The presence of the active ingredient on the PVB fibers has thus been demonstrated.

### 3. Production of Antibacterial Nanofibers Based on Nylon

Nylon 66 (N 66) was used; the repeat unit is

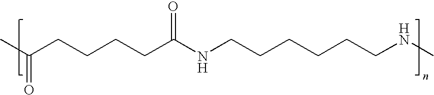

It was likewise possible to spin the polyamide solution to fibers, though the fibers produced exhibited no color whatsoever.

Nylon 66 was dissolved in formic acid with stirring at room temperature. The concentration of the solution produced was 15 wt %. In order to give the fibers an antibacterial finish, 2 wt % of quaternized PEI particles were added to the polymer solution and dispersed in the polymer solution at room temperature with stirring.

The N 66 dispersion was subsequently electrospun. The following parameters were set on the electrospinning system:

voltages: 55 kV, 60 kV
distance between cannula and electrode: 20 cm
cannula diameter: 0.3 mm
flow rates: 0.52 ml/h, 0.86 ml/h
the substrates used were aluminum foil and frames of aluminum sheet.

The average fiber diameter was 833 nm. Under an electron microscope, the fibers appear smooth as was the case for PVB, and not to have any structures on the surface, as shown by FIG. 5a and FIG. 5b.

The spider's web-like structures which can be seen in 5b are common knowledge in the spinning of nylon. In order to demonstrate the presence of the particles in the fibers, an EDX spectrum of these fibers too was recorded. Here too, iodine can be detected in the fibers, as demonstrated by the EDX spectrum in FIG. 6.

The properties of the spun nylon 66 solution are listed in Table 2.

TABLE 2
characterization of a solution of nylon 66 in formic acid used

| Nylon 66 | | | Electrical | Surface |
|---|---|---|---|---|
| concentration/ | Viscosity/ | Content of PEI | conductivity/ | tension/ |
| wt % | Pa · s | particles/wt % | μS/cm | mN/m |
| 15 | 0.659 | 0.5 | 879 | 34.62 |

### 4. Study of the Antibacterial Efficacy of the Nanofibrous Nonwoven Webs Produced

The antibacterial efficacy both of the PVB fibers and of the N 66 fibers was tested. To this end, agar plates were inoculated either with Escherichia coli or with Micrococcus luteus, admixed with an appropriate nutrient medium and incubated to confluence.

Subsequently, samples of the PVB and N 66 fiber mats comprising PEI nanoparticles were applied to the confluent E. coli and M. luteus cells and incubated at room temperature for a further 24 h. Subsequently, the effect of the fibers on the growth of the bacteria was determined with a camera.

### 5. Antibacterial Efficacy of PVB Fibers

Fiber mats were produced from PVB with a proportion of 13% by weight of PEI nanoparticles. The antibacterial efficacy was tested as stated under 4. against E. coli and M. luteus.

Neither E. coli nor M. luteus cells can exist on the PVB fiber mats, and a bacteria-free zone forms around the fiber mats. In the case of M. luteus, the bacteria-free zone is even significantly greater than that for E. coli, even though M. luteus is generally the more resistant of the two bacteria.

The antibacterial efficacy of the PVB fiber mats with 13% by weight of PEI nanoparticles on the two bacterial strains is shown in FIG. 7a (E. coli) and 7b (M. luteus).

However, a problem with PVB fibers is that they tend to degenerate under moist humid conditions as exist, for example, in the cultivation of the bacterial strains mentioned. PVB fibers which contain PEI nanoparticles are therefore suitable in particular for those purposes in which a relatively brief (single) but wide-area and strong antimicrobial action is desired.

### 6. Antibacterial Efficacy of N 66 Fibers

Fiber mats were produced from N 66 with a proportion of 13% by weight of PEI nanoparticles. The antibacterial efficacy was tested as stated under 4. against E. coli and M. luteus.

In the case of N 66 fibers with 13% by weight of PEI nanoparticles, no bacteria-free zone forms when the fibers are tested against Escherichia coli (FIG. 8a). When the fiber mat, however, is raised, a bacteria-free area can be seen (FIG. 8b), whose shape corresponds exactly to that of the fiber mat which lay there beforehand.

In the test against Micrococcus luteus, a bacteria-free zone forms around the fiber mat (FIG. 8c).

Even though the fiber mats made of nylon cannot degenerate, they still have antibacterial action against Micrococcus luteus and Escherichia coli. The antimicrobial action arises here essentially through the biocidal action of the fiber surfaces and not through release of the PEI particles from the fibers as in the case of the PVB-based fibers. Surprisingly, the phenomenon of the bacteria-free zone also occurs with the N 66 fibers in the test with Micrococcus luteus. However, this can only happen when particles can diffuse out of the fibers. Owing to the significantly lower fiber degeneration in the polyamide fibers, this means that Micrococcus luteus is much more sensitive to the PEI particles than Escherichia coli.

### 7. Series Study of Microbicidal Action of Different Proportions of PEI Particles in N 66 Fibers on the Growth of Micrococcus luteus and Escherichia coli

In order to quantify the microbicidal efficacy of the N 66 fibers comprising PEI nanoparticles on the growth of Escherichia coli and Micrococcus luteus, a test series was carried out with different proportions of PEI particles in the N 66 fibers. The solutions shown in tab. 3 were spun to nanofibers, and then the fiber mats were tested as described under 4. for their efficacy against Escherichia coli and Micrococcus luteus.

TABLE 3
efficacy of fibers made from a solution of 15 wt % of N 66 in
formic acid with a variable proportion of PEI particles

| Content of PEI | Content of PEI | | |
|---|---|---|---|
| particles in the | particles in the | Efficacy against | Efficacy against |
| solution/wt % | fibers/wt % | Escherichia coli | Micrococcus luteus |
| 0 | 0 | no | no |
| 0.17 | 1.1 | no | partial |
| 0.2 | 1.3 | no | yes |
| 0.6 | 4 | no | yes |
| 1 | 6 | no | yes |
| 2 | 13 | yes | yes |

The results shown in Table 3 are illustrated in FIG. 9.

Since the antibacterial action of the nanofibers against Escherichia coli is only visible when the fiber mat is removed from the bacterial lawn, all fiber mats which were tested against Escherichia coli were removed from the bacterial lawn in order to study the efficacy. As expected, the fibers without particles exhibit no biocidal action whatsoever. Bacteria grow under the fibers, and the fibers which were tested against Micrococcus luteus did not even have to be raised since the intense yellow bacterial lawn is visible through the fiber mat. Overall, the test against Escherichia coli always gave a negative result; bacterial lawns were found under all fiber mats. Only the first concentration tested, of 13 wt % of PEI particles, showed an antibacterial effect against Escherichia coli. The limit for effective use of the particles against Escherichia coli is thus a proportion of 13 wt %. In the case of Micrococcus luteus, the test in most cases had a positive result. At a proportion of 6 wt % of particles in the fibers, a bacteria-free zone formed; at proportions of 4 wt % and 1.3 wt %, the area in which the fiber mats lay on the nutrient medium remained entirely bacteria-free.

At a proportion of 1.1 wt % of PEI particles, an exact statement regarding the efficacy of the fibers against Micrococcus luteus is difficult. Although section B2 in FIG. 9 shows that a bacteria-free zone exists under the fibers, a more detailed consideration in FIG. 10 shows that yellow bacteria adhere to the fibers, and so firmly that they were not removed by turning the fiber mat over. Such a firmly adhering biofilm can arise only when the bacteria grow on the fibers themselves. Such growth of bacteria on the fiber mats cannot be found in the fiber mats at the higher concentrations of PEI particles.

For this reason, this sample is considered to have only limited efficacy against Micrococcus luteus. The limit for effective use of the fibers against Micrococcus luteus is thus at a particle content of at least 1.3 wt %.

### LIST OF REFERENCE NUMERALS

1 Voltage source
2 Capillary die
3 Syringe
4 Polyelectrolyte solution
5 Counterelectrode
6 Fiber formation
7 Fiber mat

### FIGURE LEGENDS

FIG. 1

FIG. 1 shows a schematic illustration of an apparatus suitable for performing the electrospinning process according to the invention.

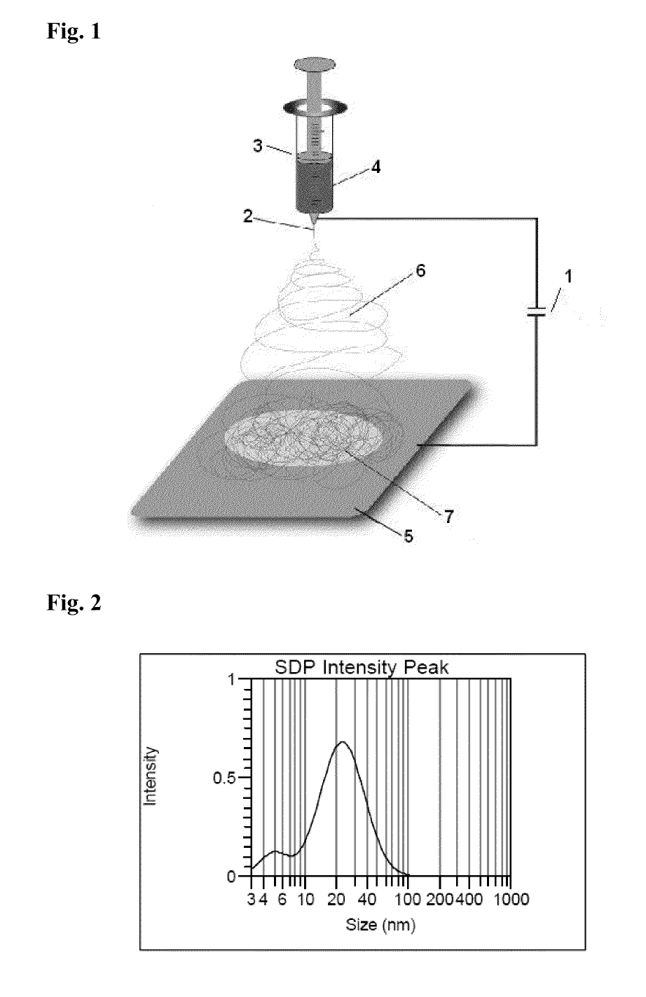

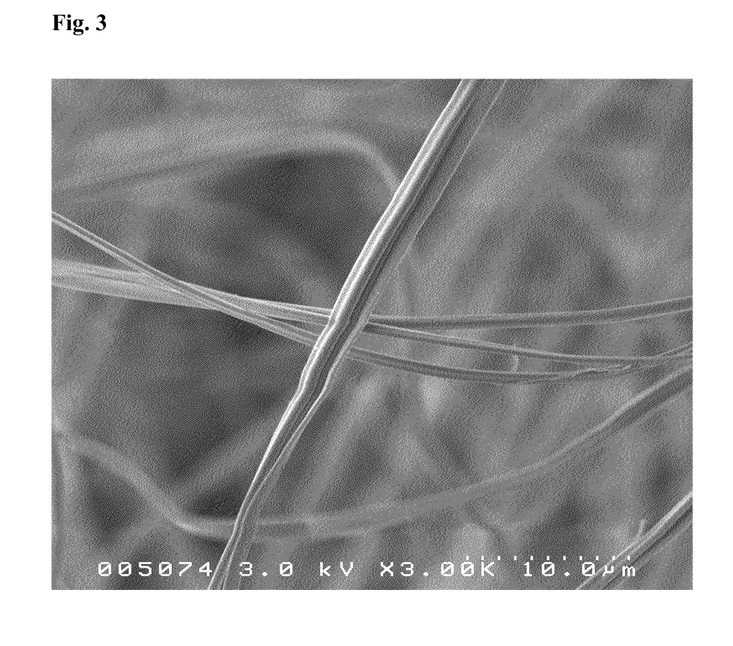

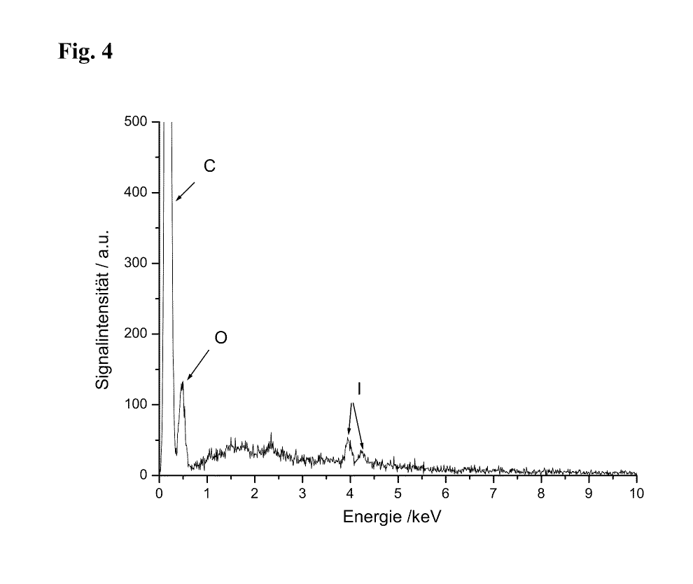

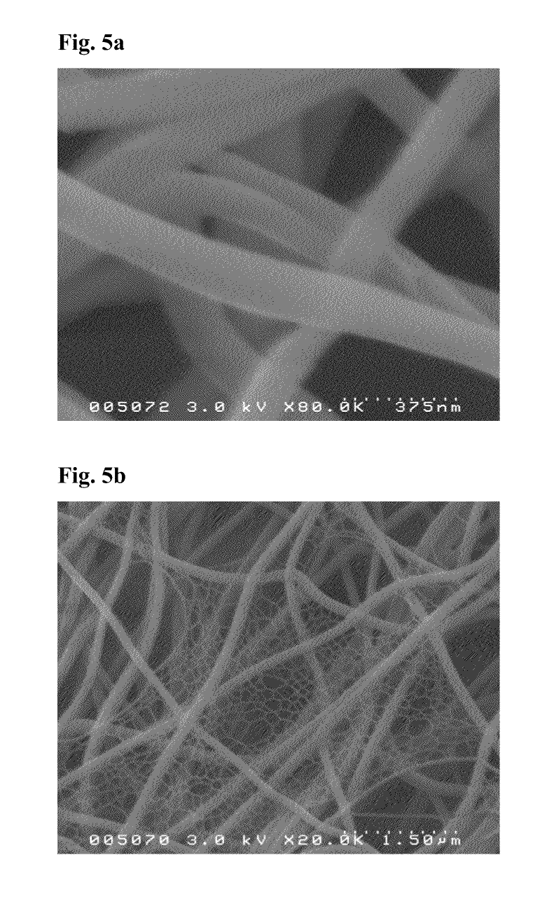

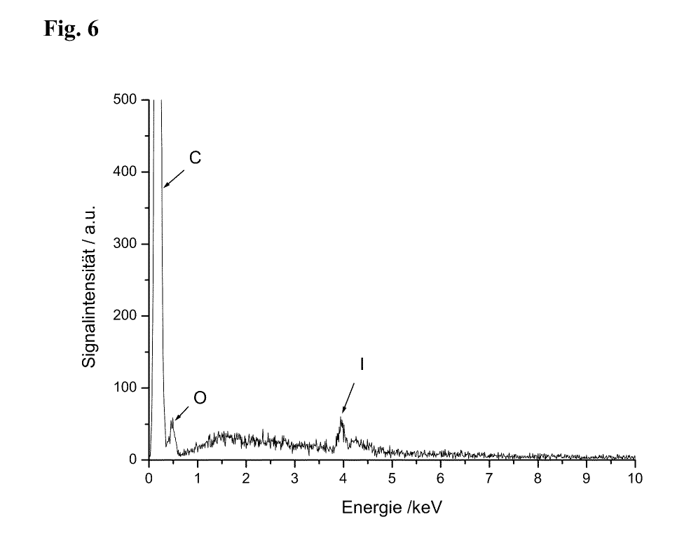

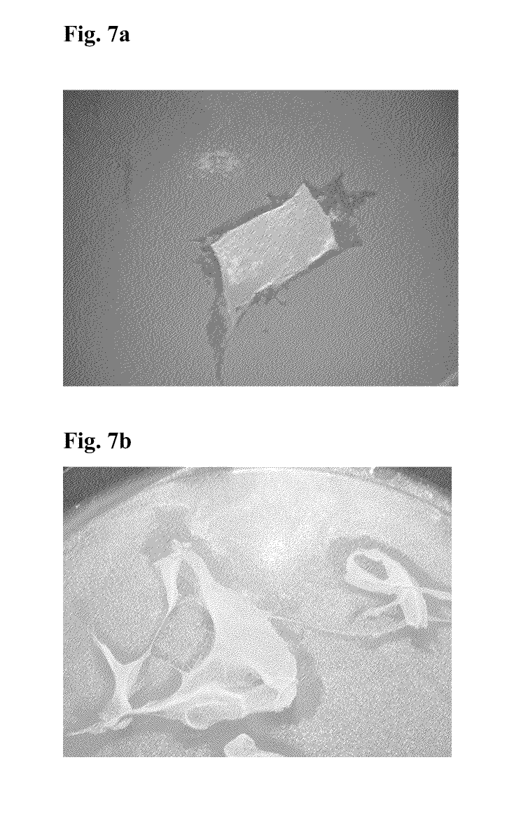

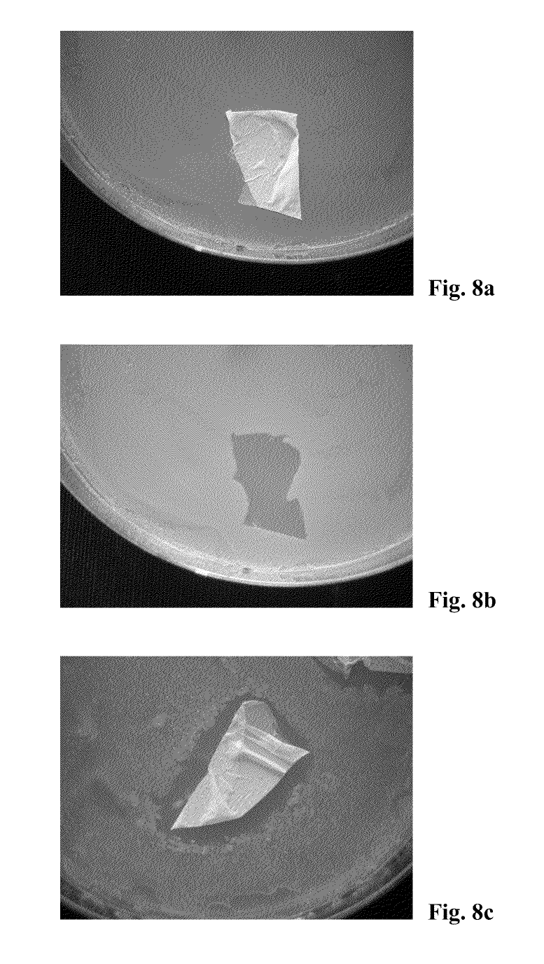

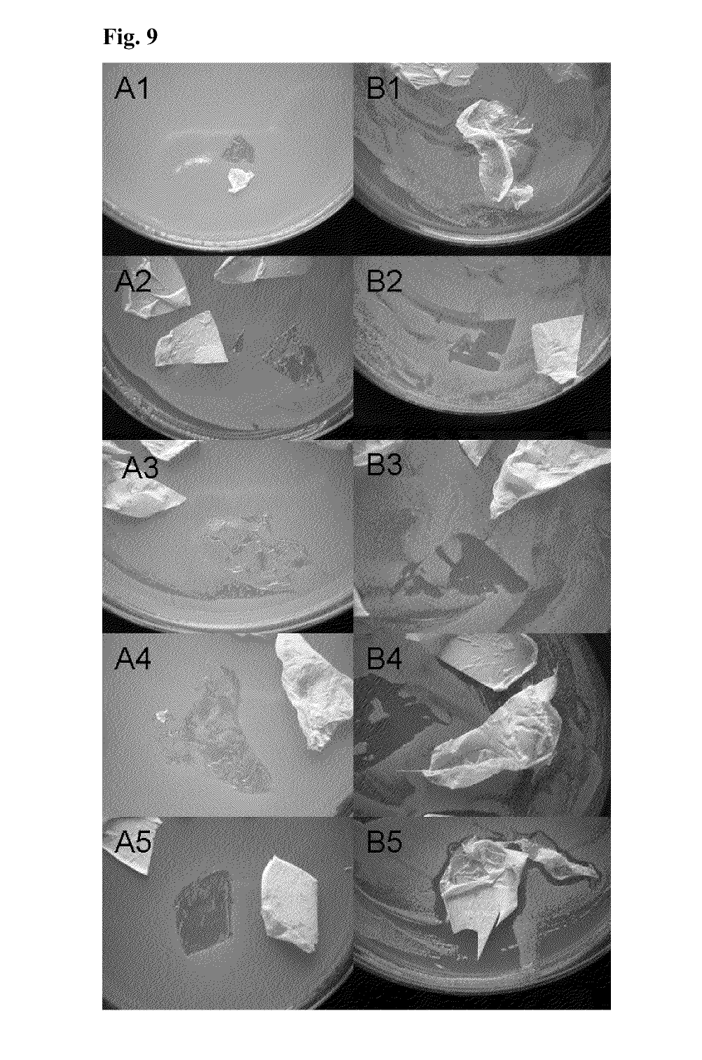

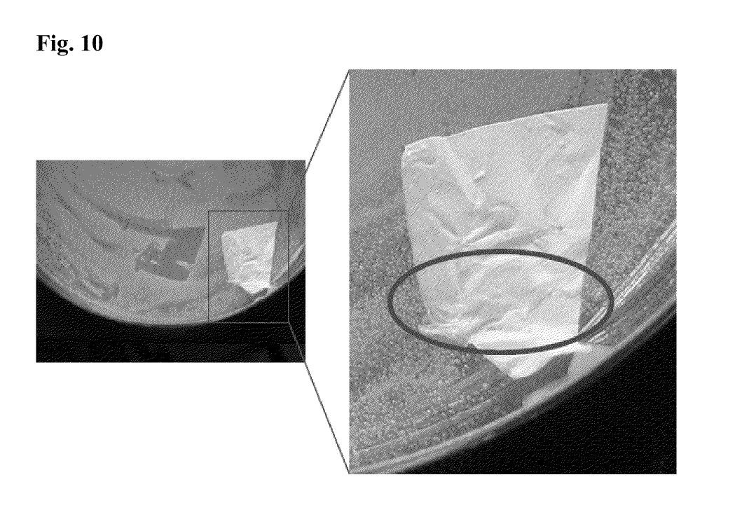

The apparatus comprises a syringe 3 at whose tip is a capillary die 2. This capillary die 2 is connected to one pole of a voltage source 1. The syringe 3 accommodates the polyelectrolyte solutions 4 to be spun. Opposite the exit of the capillary die 2 is arranged, at a distance of about 20 cm, a counterelectrode 5 which is connected to the other pole of the voltage source 1 and functions as a collector for the fibers formed. During the operation of the apparatus, a voltage between 18 kV and 35 kV is established at electrodes 2 and 5, and the polyelectrolyte solution 4 is discharged under a low pressure through the capillary die 2 of the syringe 3. Owing to the electrostatic charging of the polyelectrolytes in the solution, which is a result of strong electrical field of 0.9 to 2 kV/cm, there is a material flow directed toward the counterelectrode 5, which solidifies on the way to the counterelectrode 5 with fiber formation 6, as a result of which fibers 7 with diameters in the micro- and nanometer range are deposited on the counterelectrode 5.

FIG. 2

FIG. 2 shows the size distribution of the quaternized PEI particles. The particles were dispersed beforehand in ethanol, then the size distribution was determined with the aid of the dynamic light scattering. The average size (diameter) of the particles is about 20 nm.

FIG. 3

Fibers formed from 15% by weight of PVB in ethanol, with 2% by weight of PEI particles, SEM image, 8000-fold magnification.

FIG. 4

EDX spectrum of a fiber mat formed from PVB admixed with 2% by weight of PEI particles, acceleration voltage 20 kV.

FIG. 5

Fibers formed from a solution of 15% by weight of N 66 in formic acid with an addition of 0.5% by weight of PEI;
a) SEM image, 8000-fold magnification,
b) SEM image, 20 000-fold magnification.

FIG. 6

EDX spectrum of fiber mats formed from a solution of 15% by weight of N 66 in formic acid with 2% by weight of PEI particles.

FIG. 7

Fiber mats formed from PVB with a proportion of 13% by weight of PEI nanoparticles in the fibers,
a) laid onto a confluent layer of Escherichia coli, incubated at room temperature for 24 h
b) laid onto a confluent layer of Micrococcus luteus, incubated at room temperature for 24 h

FIG. 8

Fiber mats formed from N 66 with a proportion of 13% by weight of PEI nanoparticles in the fibers,
a) laid onto a confluent layer of Escherichia coli, incubated at room temperature for 24 h
b) laid onto a confluent layer of Escherichia coli, incubated at room temperature for 24 h after raising the fiber mat,
c) laid onto a confluent layer of Micrococcus luteus, incubated at room temperature for 24 h

FIG. 9

Series study of the efficacy of N 66 fibers with different proportions of PEI particles.

Series A: tested on Escherichia coli; all fiber mats were raised.
a) A1) no PEI particles,
b) A2) 1.1 wt % of particles,
c) A3) 1.3 wt % of particles,
d) A4) 4 wt % of particles,
e) A5) 6 wt % of particles.

Series B: tested against Micrococcus luteus, fiber mats B2 to B4 were raised.
f) B1) no PEI particles,
g) B2) 1.1 wt % of particles,
h) B3) 1.3 wt % of particles,
i) B4) 4 wt % of particles,
j) B5) 6 wt % of particles.

FIG. 10

Enlargement of the image section B2 from FIG. 9:

the efficacy of N 66 fibers with 1.1 wt % of PEI particles was tested here on the growth of Micrococcus luteus; the fiber mat was raised. Bacteria adhere to the fibers, and so firmly that they are not removed by turning the fiber mat over.

1. A polymeric fiber having microbicidal properties, comprising at least one electrospinnable polymer and nanoparticles comprising derivatized polyethyleneimine, wherein the polymeric fiber is an electrospun polymeric fiber.

2. The polymeric fiber of claim 1 wherein the derivatized polyethyleneimine comprises quaternized polyethyleneimine.

3. The polymeric fiber of claim 1 wherein the electrospinnable polymer is selected from the group consisting of poly-(p-xylylene); polyvinylidene halides; polyesters; polyethers; polyolefins; polycarbonates; polyurethanes; natural polymers; polycarboxylic acids; polysulfonic acids; sulfated polysaccharides; polylactides; polyglycosides; polyamides; homo- and copolymers of aromatic vinyl compounds; polyacrylonitriles, polymethacrylonitriles; polyacrylamides; polyimides; polyphenylenes; polysilanes; polysiloxanes; polybenzimidazoles; polybenzothiazoles; polyoxazoles; polysulfides; polyester amides; polyarylene vinylenes; polyether ketones; polyurethanes; polysulfones; inorganic-organic hybrid polymers; silicones; wholly aromatic copolyesters; poly(alkyl)acrylates; poly(alkyl)methacrylates; polyhydroxyethyl methacrylates; polyvinyl acetates; polyvinyl butyrates; polyisoprene; synthetic rubbers; polytetrafluoroethylene; modified and unmodified celluloses; homo- and copolymers of alpha-olefins and copolymers constructed of two or more monomer units forming the aforementioned polymers; polyvinyl alcohols, polyalkylene oxides; poly-N-vinylpyrrolidone; hydroxymethylcelluloses; maleic acids; alginates; and collagens.

4. The polymeric fiber of claim 1, wherein the nanoparticles comprise quaternized polyethyleneimine of the general formula 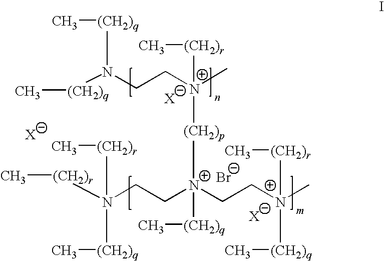
where
m and n are each independently a natural number from 5 to 200,
p is a natural number from 4 to 6,
q is a whole number from 0 to 11,
r is a whole number from 0 to 4 and
X is Br or I.

5. The polymeric fiber of claim 1 wherein the proportion of polyethyleneimine nanoparticles in the fiber is between 0.1 wt % to 25 wt %.

6. A process for producing microbicidal polymeric fiber as claimed in claim 1, comprising the steps of:
a) crosslinking polyethyleneimine,
b) alkylating crosslinked polyethyleneimine,
c) quaternizing secondary and tertiary amino groups of the polyethyleneimine,
d) removing the quaternized polyethyleneimine nanoparticles,
e) adding the polyethyleneimine nanoparticles to a solution of one or more electrospinnable polymers, and
f) electrospinning the solution of one or more electrospinnable polymers which contains polyethyleneimine nanoparticles to form fiber.

7. A process for producing polymeric fiber as claimed in claim 1, comprising the steps of:
a) crosslinking polyethyleneimine,
b) alkylating crosslinked polyethyleneimine,
c) quaternizing secondary and tertiary amino groups of the polyethyleneimine,
d) removing the quaternized polyethyleneimine nanoparticles,
e) electrospinning a solution of at least one electrospinnable polymer to form fiber, and
f) coating the electrospun fiber with polyethyleneimine nanoparticles.

8. A process for producing polymeric fiber as claimed in claim 1, comprising the steps of:
a) crosslinking polyethyleneimine,
b) alkylating crosslinked polyethyleneimine,
c) quaternizing secondary and tertiary amino groups of the polyethyleneimine,
d) removing the quaternized polyethyleneimine nanoparticles,
e) adding the polyethyleneimine nanoparticles to a solution of one or more electrospinnable polymers,
f) electrospinning the solution of one or more electrospinnable polymers which contains polyethyleneimine nanoparticles to form fiber, and
g) coating the electrospun fiber with polyethyleneimine nanoparticles.

9. (canceled)

10. A textile fabric comprising a microbicidal polymeric fiber of claim 1.

11. An article of manufacture comprising a textile fabric of claim 10, wherein the article of manufacture is selected from the group consisting of: functional apparel, protective apparel for medical personnel, protective apparel for patients, surgical drapes, wound dressings, and fibrous nonwoven webs and fibrous mats for cell culture substrates.
